(19) United States
(12) Patent Application Publication
(10) Pub. No.: US 2025/0050064 A1
(43) Pub. Date: Feb. 13, 2025

(54) BORE EMBOLIZATION CATHETER

(71) Applicant: The University of Toledo, Toledo, OH (US)

(72) Inventors: Syed F. Zaidi, Toledo, OH (US); Mouhammad A. Jumaa, Toledo, OH (US)

(73) Assignee: The University of Toledo, Toledo, OH (US)

(21) Appl. No.: 18/719,550

(22) PCT Filed: Dec. 15, 2022
(86) PCT No.: PCT/US2022/052929

§ 371 Date: Jun. 13, 2024

Related U.S. Application Data
(60) Provisional application No. 63/290,904, filed on Dec. 17, 2021.

(51) Int. Cl. A61M 25/00

(52) U.S. Cl. CPC A61M 25/0074

(57) ABSTRACT

A bore embolization catheter comprising a catheter main body extending from a catheter junction to a distal end and housing a first lumen, a second lumen, and a third lumen; a detachment zone on the catheter main body configured to allow the catheter main body to be broken; an agent delivery hole on the catheter main body between the detachment zone and the distal end; an end port at the distal end; an inflatable balloon on the catheter main body between the agent delivery hole and the distal end; and a first port in communication with the inflatable balloon through the first lumen, a second port in communication with the agent delivery hole through the second lumen, and a third port in communication with the end port through the third lumen.

### RELATED APPLICATIONS

This application claims priority to U.S. Provisional Application No. 63/290,904, filed under 35 U.S.C. § 111 (b) on Dec. 17, 2021, the entire disclosure of which is expressly incorporated herein by reference for all purposes.

### STATEMENT REGARDING FEDERALLY SPONSORED RESEARCH

This invention was made with no government support. The government has no rights in this invention.

### BACKGROUND

Arteriovenous malformations (AVMs) are abnormal connections between arteries and veins in the brain or spine. The distinguishing feature of an AVM is that shunting occurs through a collection of tortuous dysmorphic vessels, referred to as a nidus. AVMs are difficult or dangerous to treat and may cause bleeding into or around the brain. Treatment for an AVM may involve partial or complete closure of the AVM through embolization (blocking).

Currently, AVM embolization techniques include direct embolization using a traditional microcatheter, balloon guided embolization, and a pressure cooker technique using two individual microcatheters. The current treatment paradigm entails placement of either a 1.3″ Fr or 1.7″ Fr microcatheter in close approximation to the vascular malformation and administration of a liquid embolic agent into the malformation. Frequently, however, the embolic agent tends to reflux backwards instead of moving distally into the lesion. To prevent this reflux and augment distal penetration of the embolic agent, a “pressure cooker” technique has been described. The pressure cooker technique involves placement of another microcatheter proximal to the original treatment microcatheter and the administration of a liquid embolic agent from it. However, in many instances, this is not sufficient to prevent reflux of the embolic agent. Thus, there is a need in the art for new and improved devices and methods for accomplishing AVM embolization.

### SUMMARY

Provided herein is a bore embolization catheter comprising a catheter main body extending from a catheter junction to a distal end and housing a first lumen, a second lumen, and a third lumen; a detachment zone on the catheter main body configured to allow the catheter main body to be broken; an agent delivery hole on the catheter main body between the detachment zone and the distal end; an end port at the distal end; an inflatable balloon on the catheter main body between the agent delivery hole and the distal end; and a first port, a second port, and a third port extending from the catheter junction and defining a proximal end, wherein the first port is in communication with the inflatable balloon through the first lumen, the second port is in communication with the agent delivery hole through the second lumen, and the third port is in communication with the end port through the third lumen.

In certain embodiments, the bore embolization catheter is a microcatheter, the catheter main body having a diameter ranging from 0.70 mm to 1.30 mm.

In certain embodiments, the catheter junction joins the first port, the second port, and the third port. In certain embodiments, the catheter junction is between the detachment zone and the proximal end. In certain embodiments, the detachment zone is between the catheter junction and the agent delivery hole.

In certain embodiments, the agent delivery hole is between the inflatable balloon and the detachment zone.

In certain embodiments, the detachment zone includes a detachment hole.

In certain embodiments, the bore embolization catheter comprises a plurality of agent delivery holes. In certain embodiments, the bore embolization catheter comprises six agent delivery holes all disposed between the inflatable balloon and the detachment zone.

Further provided is a method of using the bore embolization catheter described herein, the method comprising inserting the bore embolization catheter into a feeding artery; guiding the bore embolization catheter through the feeding artery and into an inflow vessel having an AVM nidus located therein; positioning the bore embolization catheter between the AVM nidus and a normal vessel; inflating the inflatable balloon within the inflow vessel by applying a fluid through the first port to form an inflated balloon; delivering an embolic agent to occlude the inflow vessel proximally to the inflated balloon by administering the embolic agent into the second port and through the agent delivery hole; and treating the AVM nidus by administering a treatment substance into the third port and through the end port, wherein the treatment substance is prevented from flowing into the inflow vessel by the embolic agent. In certain embodiments, the method further comprises draining fluids near the distal end through the end port. In certain embodiments, the method further comprises breaking the bore embolization catheter at the detachment zone.

Further provided is a bore embolization catheter comprising a catheter main body extending from a catheter junction to a distal end and housing a first lumen and a second lumen; a detachment zone on the catheter main body configured to allow the catheter main body to be broken; an agent delivery hole between the detachment zone and the distal end; an end port at the distal end; and a first port and a second port extending from the catheter junction and defining a proximal end, wherein the first port is in communication with the agent delivery hole through the first lumen, and the second port is in communication with the end port through the second lumen.

In certain embodiments, the bore embolization catheter is a microcatheter, the catheter main body having a diameter ranging from 0.70 mm to 1.30 mm.

In certain embodiments, the catheter junction joins the first port and the second port. In certain embodiments, the catheter junction is between the detachment zone and the proximal end. In certain embodiments, the detachment zone is between the catheter junction and the agent delivery hole. In certain embodiments, the agent delivery hole is between the detachment zone and the distal end.

In certain embodiments, the detachment zone includes a detachment hole.

In certain embodiments, the bore embolization catheter comprises a plurality of agent delivery holes. In particular embodiments, the bore embolization catheter comprises six agent delivery holes all disposed between the detachment zone and the distal end.

Further provided is a method of using a bore embolization catheter described herein, the method comprises inserting the bore embolization catheter into a feeding artery; guiding the bore embolization catheter into an inflow vessel having an AVM nidus therein; positioning the bore embolization catheter between the AVM nidus and the normal vessel; delivering an embolic agent into the first port and out of the agent delivery hole into the inflow vessel; and delivering a treatment substance to treat the AVM nidus by administering the treatment substance into the second port and out of the end port, wherein the treatment substance is prevented from flowing into the feeding artery by the embolic agent. In certain embodiments, the method further comprises breaking the bore embolization catheter at the detachment zone. In certain embodiments, the method further comprises draining fluids near the distal end through the end port.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1: Perspective view of an embodiment of a bore embolization catheter that includes an inflatable balloon.

FIG. 2: Perspective view of the bore embolization catheter situated within a feeding artery and an inflow vessel.

FIG. 3: Perspective view of the bore embolization catheter situated within an inflow vessel nearby an AVM nidus.

FIG. 4: Perspective view of the bore embolization catheter situated within the inflow vessel deploying an embolic agent.

FIG. 5: Sectional view of an interior portion of the bore embolization catheter.

FIG. 6: Perspective view of another embodiment of a bore embolization catheter which does not include an inflatable balloon.

FIG. 7: Perspective view of the bore embolization catheter situated within a feeding artery and an inflow vessel.

FIG. 8: Perspective view of the bore embolization catheter situated within the inflow vessel nearby an AVM nidus.

FIG. 9: Perspective view of the bore embolization catheter situated within the inflow vessel deploying an embolic agent.

FIG. 10: Sectional view of an interior portion of the bore embolization catheter.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein is a bore embolization catheter configured to apply one or more agents, or substances, to an area or malformation within a blood vessel. The bore embolization catheter can be used for a multitude of purposes including, but not limited to, applying an embolic agent in an inflow vessel between an AVM nidus and a normal vessel, applying an embolic agent within a vessel where there is no AVM nidus present, applying an embolic agent in a feeding vessel, or administering a sclerosant solution to a vessel malformation. Advantageously, rather than applying the embolic agent from only a distal end of the bore embolization catheter, the embolic agent can be deployed from at least one agent delivery hole disposed a distance proximally away from the distal end of the bore embolization catheter. The bore embolization catheter also allows for application of an embolic agent without the embolic agent approaching the normal vessels. In some embodiments, the bore embolization catheter is a microcatheter with an additional proximal port to augment delivery of liquid embolic agent into intracranial vascular malformations so as to prevent reflux of the embolic agent. Also provided is a method for using the embolization catheter to apply an embolic agent in the inflow vessel between a normal vessel and an AVM nidus. The ability to administer the embolic agent in this manner minimizes the chances that the embolic agent will leak into the normal vessels that are not in need of treatment. In some embodiments, using the bore embolization catheter for the application of an embolic agent and/or a sclerosant solution provides a way of effectively treating an AVM nidus without damaging or risking other vessels in proximity to where the embolic agent and/or sclerosant solution is released.

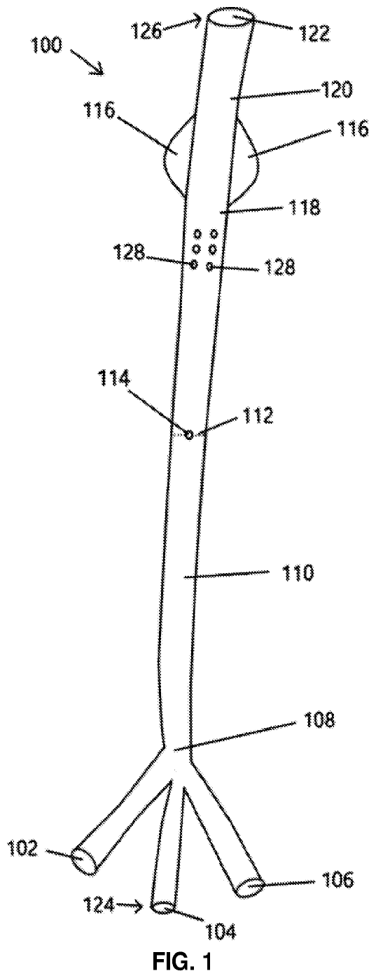

Referring now to FIG. 1, a first embodiment of a bore embolization catheter 100 is depicted. The bore embolization catheter 100 may be a microcatheter, having a catheter main body 110 with a diameter ranging from 0.70 mm to 1.30 mm. However, other sizes are possible and encompassed within the scope of the present disclosure. The bore embolization catheter 100 includes a catheter main body 110 extending from a catheter junction 108 to a distal end 126 of the bore embolization catheter 100. Extending from the catheter junction 108, the bore embolization catheter 100 includes a plurality of ports 102, 104, 106 which extend further proximally. The ports 102, 104, 106 define openings which provide access to internal lumens 172, 174, 176, as discussed in more detail below.

Referring still to FIG. 1, the bore embolization catheter 100 may include a first port 102, a second port 104, and a third port 106 extending proximally from the catheter junction 108. The first port 102, second port 104, and third port 106 are joined together in the catheter junction 108 to form the catheter main body 110. The bore embolization catheter 100 has a proximal end 124 defined by the ends of the first port 102, the second port 104, and the third port 106.

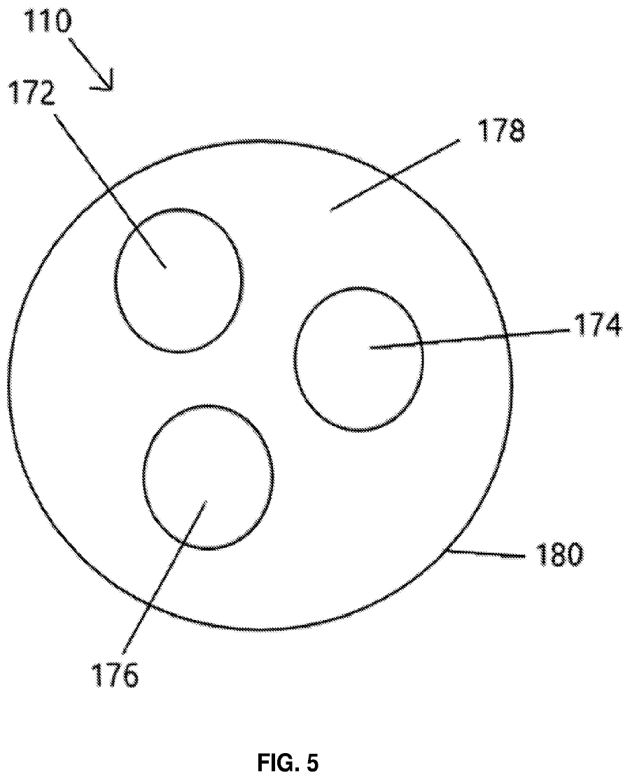

Referring now to FIG. 5, depicted is a sectional view of the catheter main body 110, a perimeter of which is defined by a catheter wall 180. The catheter wall 180 bounds a catheter interior 178, in which a first lumen 172, a second lumen 174, and a third lumen 176 are disposed. The first port 102 feeds into, and is in communication with, the first lumen 172 within the catheter main body 110. The second port 104 feeds into, and is in communication with, the second lumen 174 within the catheter main body 110. The third port 106 feeds into, and is in communication with, the third lumen 176 within the catheter main body 110. Thus, when a substance is introduced into the first port 102, the substance may flow into the first lumen 172; when a substance is introduced into the second port 104, the substance may flow into the second lumen 174; and when a substance is introduced into the third port 106, the substance may flow into the third lumen 176.

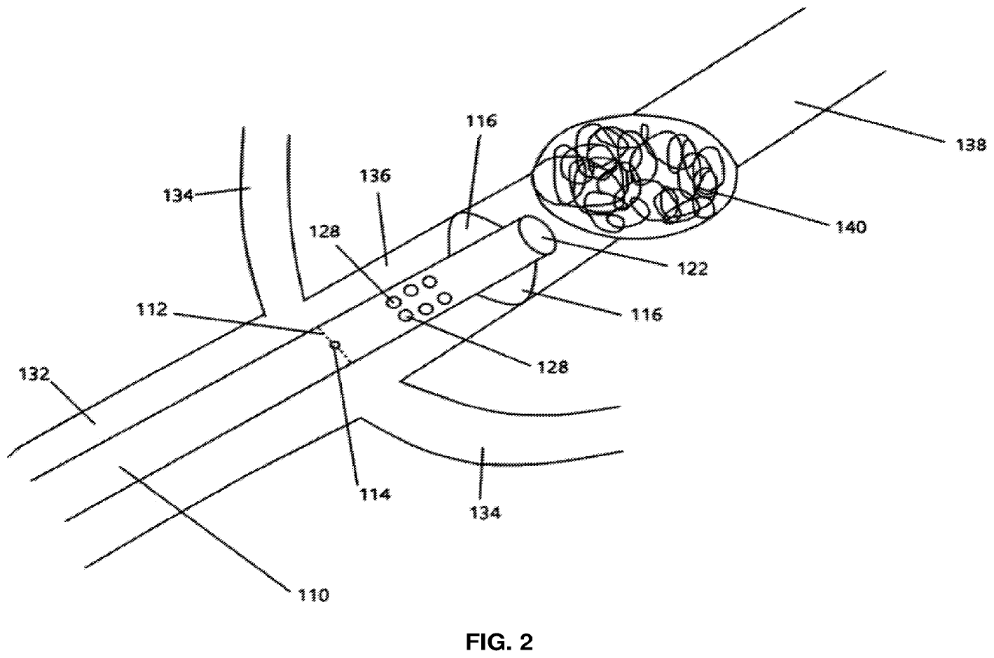

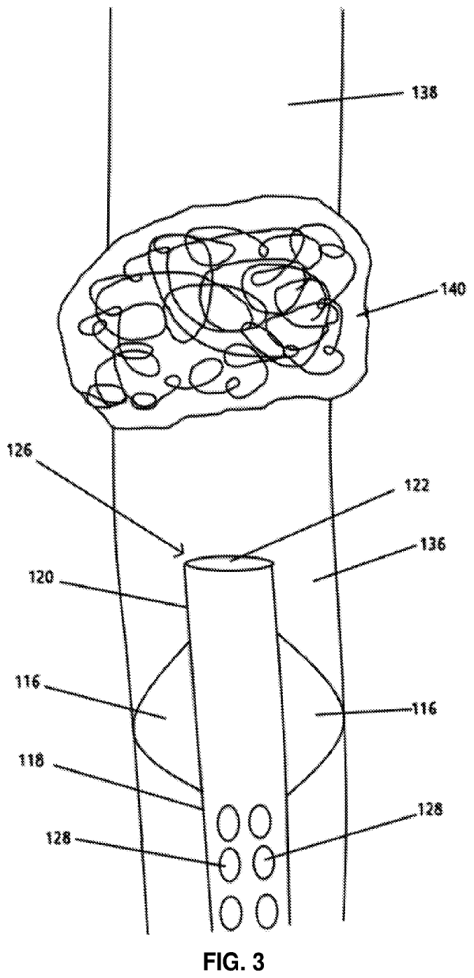

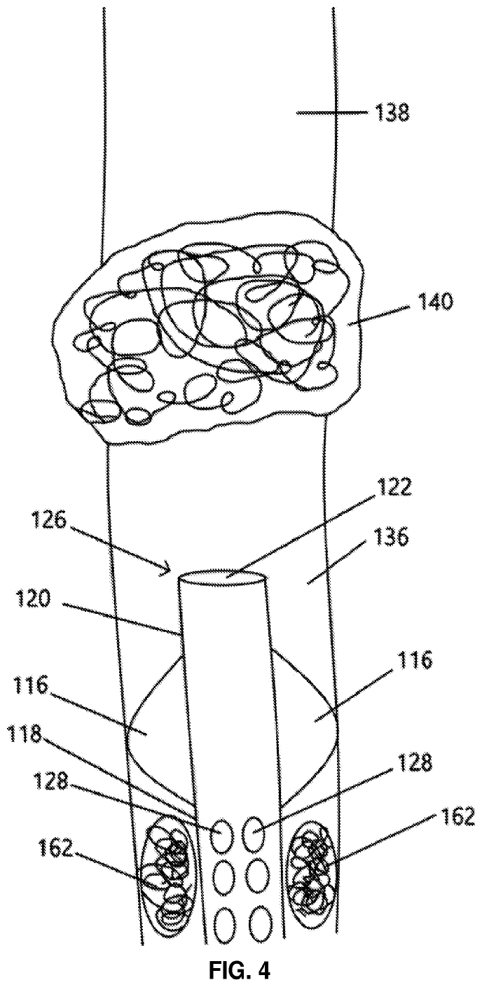

Referring to FIGS. 1-5, the first lumen 172 extends through the catheter main body 110 and feeds into, and is in communication with, an inflatable balloon 116. The inflatable balloon 116 may be disposed around the catheter main body 110 near, but removed from, the distal end 126 of the bore embolization catheter 100. The inflatable balloon 116 may be inflated by providing a fluid, such as air, water, or any other type of gas or liquid, into the first port 102, through the first lumen 172, and into the inflatable balloon 116. The inflatable balloon 116 may be inflated to a desired size. As depicted in FIG. 2, the inflatable balloon 116 may be utilized to substantially occlude a vessel 136. Thus, the inflatable balloon 116 may be inflated to a size substantially the same as the internal diameter of a blood vessel 136. Advantageously, the inflatable balloon 116 allows for a vessel 136 to be blocked off to prevent a medication, or treatment substance, from flowing in the wrong direction (i.e., prevent reflux), such as away from an AVM nidus 140 or other malformation to be treated. The inflatable balloon 116 can also be inflated for the purpose of restricting the flow of fluids through the vessel 136 so the substances delivered through the end port 122 and/or the agent delivery holes 128 do not enter or block normal vessels 134. Normal vessels 134, in this context, refer to vessels which do not have a blockage, AVM nidus 140, or any other vessel malformation. The inflatable balloon 116 may also ensure that a proximal plug of embolic agent 162 delivered through the agent delivery holes 128 remains localized within the vessel 136. Thus, as shown in FIG. 4, the inflatable balloon 116 prevents distal migration of the embolic agent 162.

Referring again to FIGS. 1-5, the second lumen 174 extends through the catheter main body 110 and feeds into, and is in communication with, one or more agent delivery holes 128. The agent delivery holes 128 are holes in the catheter main body 110 configured to allow passage of a substance therethrough, i.e., from the second lumen 174 out of the catheter main body 110 and into an area adjacent to the bore embolization catheter 100. The agent delivery holes 128 may number from four to eight in some embodiments. In some embodiments, the bore embolization catheter 100 includes six agent delivery holes 128, as this number is useful for deploying an adequate amount of embolic agent 162 in a desired location without the embolic agent 162 spreading too far from the desired location. However, the number of agent delivery holes 128 is not particularly limited, and other numbers of agent delivery holes 128 are possible and encompassed within the scope of the present disclosure. Non-limiting examples of the embolic agent 162 that can be deployed through the agent delivery holes 128 include, but are not limited to, polyvinyl alcohol (PVA), tris-acryl gelatin microspheres, sclerosant, ethanol, polymers, N-butyl cyanoacrylate (NBCA) glue, or ethylene vinyl alcohol. However, other embolic agents, such as a glue substance, a powder substance, or any accepted materials that are normally put into a vessel, may be utilized and delivered through the agent delivery holes 128.

Referring still to FIGS. 1-5, the agent delivery holes 128 are disposed proximally to the inflatable balloon 116, and are therefore a greater distance away from the distal end 126 than the inflatable balloon 116 is from the distal end 126. The agent delivery holes 128 are also between the catheter junction 108 and the inflatable balloon 116, and are therefore closer to the proximal end 124 of the bore embolization catheter 100 than the inflatable balloon 116 is to the proximal end 124. In one non-limiting example, the agent delivery holes 128 are disposed about 2.5 cm proximal to the distal end 126. As seen in FIG. 4, the agent delivery holes 128 can be used to deliver an embolic agent 162 to a location within a vessel 136 a distance away from an AVM nidus 140, and in a location such that a treatment can be delivered to the AVM nidus 140 through the end port 122 in a space between the delivered embolic agent 162 and the AVM nidus 140. The embolic agent 162 can form a proximal plug within the vessel 136, and the position of the inflatable balloon 116 relative to the agent delivery holes 128 may ensure that the proximal plug of embolic agent 162 remains localized, i.e., does not migrate distally, thereby negating the purpose of the dual lumen microcatheter 100.

To deliver an embolic agent 162 through the agent delivery holes 128, the embolic agent 162 can be introduced into the second port 102, and flow through the second lumen 174 and out the agent delivery holes 128 into the vessel 136. Advantageously, the location of the agent delivery holes 128 being proximate to the inflatable balloon 116 allows for augmented delivery of the embolic agent 162. It also allows, if desired, for the inflatable balloon 116 to be included in the portion of the bore embolization catheter 100 that is broken off and left behind in a vessel 136, as described in more detail below.

Referring again to FIGS. 1-5, the third lumen 176 extends through the catheter main body 110 and feeds into, and is in communication with, the end port 122 at the distal end 126 of the bore embolization catheter 100. A medication or other substance useful for treating an AVM nidus 140 or other malformation may be introduced into the third port 106 and flow through the third lumen 176 and out of the end port 122 at the distal end 126 of the bore embolization catheter 100. Thus, third port 106, third lumen 176, and end port 122 may be used to administer a substance such as a sclerosant solution to an area within a vessel 136 needing treatment, such as an area adjacent to an AVM nidus 140. Non-limiting examples of sclerosant solutions include hypertonic saline (HS), hypertonic dextrose, sodium salicylate, and a combination of HS and hypertonic dextrose. The end port 122 can also be used to deliver any other type of substance that can shrink the vessel 136 and/or cause scars to form. As depicted in FIG. 4, the distal end 126 may be disposed between the AVM nidus 140 and the inflatable balloon 116, effectively creating a sealed off area to prevent backflow of the treatment substance when the inflatable balloon 116 is inflated to a diameter approximately the diameter of the vessel 136.

Notwithstanding the above description of the first lumen 172, the second lumen 174, and the third lumen 176, it is understood that the lumens 172, 174, 176 may be switched or reversed in any configuration. In other words, it is possible, for example, for the first lumen 172 to connect the first port 102 to the end port 122, or of the second lumen 174 to connect the third port 106 to the agent delivery holes 128, and so on. The lumens 172, 174, 176 can also be positioned in any desired arrangement in the catheter interior 178 within the catheter main body 110. Furthermore, though the administration of an embolic agent 162 through the agent delivery holes 128 and the administration of a sclerosant solution through the end port 122 are described for exemplary purposes, the composition of any substance or drug delivered through the bore embolization catheter 100 is not particularly limited.

Referring now to FIGS. 1-2, the catheter main body 110 may include a detachment zone 112, which can be in the form of perforations, indentations, or other weak points in the material making up the catheter main body 110, and/or a detachment zone hole 114. The detachment zone 112 and/or the detachment zone hole 114 can be used for the purpose of breaking the catheter main body 110 at a desired location after a procedure has been completed or even during a procedure, as desired. The agent delivery holes 128, inflatable balloon 116, and end port 122 are all distal to the detachment zone 112 and the detachment hole 114 (i.e., are all closer to the distal end 126 than the detachment zone 112 and the detachment hole 114 are). The detachment zone 112 and/or the detachment zone hole 114 allow for a section of the bore embolization catheter 100 (i.e., the section distal to the detachment zone 112 and/or detachment zone hole 114) to remain in place within a vessel 136 instead of having to remove it. Advantageously, this can prevent inadvertently causing damage to the vessel 136 and to surrounding, normal vessels 134 from the process of the retrieving the bore embolization catheter 100 once the embolic agent 162 has been delivered. In some embodiments, the detachment zone 112 and/or detachment hole 114 may cause breakage of the catheter main 110 body upon applying a twisting and/or pulling motion to the proximal end 124 of the bore embolization catheter 100 once embolic agent 162 is situated around the agent delivery holes 162. The embolic agent 162 may adhere to the catheter main body 110 sufficiently enough to cause resistance to the movement of the catheter main body 110 in an amount sufficient to cause breakage of the catheter main body 110 at the detachment zone 112 and/or detachment hole 114.

Referring still to FIGS. 1-5, the end port 122 may also be utilized to drain fluids from within a vessel 136, such as from an area around an AVM nidus 140 or between an AVM nidus 140 and the inflatable balloon 116. Fluids can be allowed to enter the end port 122 and travel through the third lumen 176 to the third port 106 (optionally, with the aid of suction applied to the third port 106) in order to drain. Alternatively, if the catheter main body 110 has already been broken at the detachment zone 112 and/or detachment hole 114, then such fluids may drain out of the broken catheter main body 110 at the break (i.e., at the detachment zone 112 and/or detachment hole 114), which may be on the opposing side of an embolic agent 162 compared to an AVM nidus 140 or other malformation.

As discussed above, each of the first, second, and third ports 102, 104, 106 is configured to perform separate functions. However, it does not matter which port 102, 104, 106 is used for which function, and it does not matter which lumen 172, 174, 176 connects to a given port 102, 104, 106. Each of the ports 102, 104, 106 may be used for a different function. For example, the first port 102 can be used to inflate the balloon 116 by any known method of inflating the balloon 116 with air, a liquid substance, or any other type of fluid that will cause the balloon 116 to inflate. The second port 104 can be used for the delivery of an agent 162 such as, but not limited to, an embolic agent. The third port 106 can be used for the delivery of a substance, such as a medication, or any other means of treating an AVM nidus referenced as 140 in FIG. 2. However, this is also not necessary. Rather, in some embodiments, for example, multiple of the ports 102, 104, 106 may be utilized for delivering the same substance.

Referring now to FIG. 2, the bore embolization catheter 100 is depicted situated within a feeding artery 132 and within the inflow vessel 136. The feeding artery 132 is the vessel through which the bore embolization catheter 100 is fed or guided to reach the location in need of treatment. The bore embolization catheter 100 is depicted situated within the feeding artery 132 and extending past normal vessels 134 into the inflow vessel 136. The inflow vessel 136 is the vessel in which embolic agent 162 is to be delivered. Within the inflow vessel 136, the inflatable balloon 116 can be inflated before the bore embolization catheter 100 reaches the AVM nidus 140. Behind the AVM nidus 140, relative to the bore embolization catheter 100, is the outflow portion 138 of the inflow vessel 136. A portion of the bore embolization catheter 100 that includes the agent delivery hole 128 can be positioned within the inflow vessel 136, such that an embolic agent 162 can be delivered through the agent delivery holes 128, as depicted in FIG. 4, and the embolic agent 162 is present within the inflow vessel 136 and not within any other vessels.

Referring now to FIGS. 2-4, depicted is a method for applying a treatment to an AVM nidus 140 with the bore embolization catheter 100. The bore embolization catheter 100 can be used for treating irregularities or malformations in blood vessels including, but limited to, an AVM nidus 140. The method for treating an AVM nidus 140 can begin by inserting the bore embolization catheter 100 into a feeding artery 132. After inserting the bore embolization catheter 100 into the feeding artery 132, the bore embolization catheter 100 can be guided through the feeding artery 132 into an inflow vessel 136 in which the AVM nidus 140 is located. The bore embolization catheter 100 can then be positioned between the AVM nidus 140 and a normal vessel 134. The balloon 116 can then be inflated within the inflow vessel 136 by applying a fluid, such as air, water, or any other type of gas or liquid, through the first port 102. When the inflatable balloon 116 is inflated to the desired size, the embolic agent 162 can be deployed on the proximal side 118 of the catheter main body 110 relative to the balloon 116 by administering the embolic agent 162 into the second port 104, through the second lumen 174, and out the agent delivery holes 128. Finally, the treatment substance can be deployed to treat the AVM nidus 140 by administering the treatment substance into the third port 106, through the third lumen 176, and out the end port 122. Also, the third port 106 can be used to drain fluids from the distal end 126 of the bore embolization catheter 100. Once the treatment substance has been delivered, the bore embolization catheter may be broken at the detachment zone 112 or the detachment hole 114 to leave in place some of the bore embolization catheter 100, with embolic agent 162 around it, and the remaining portion of the bore embolization catheter 100 can be retrieved.

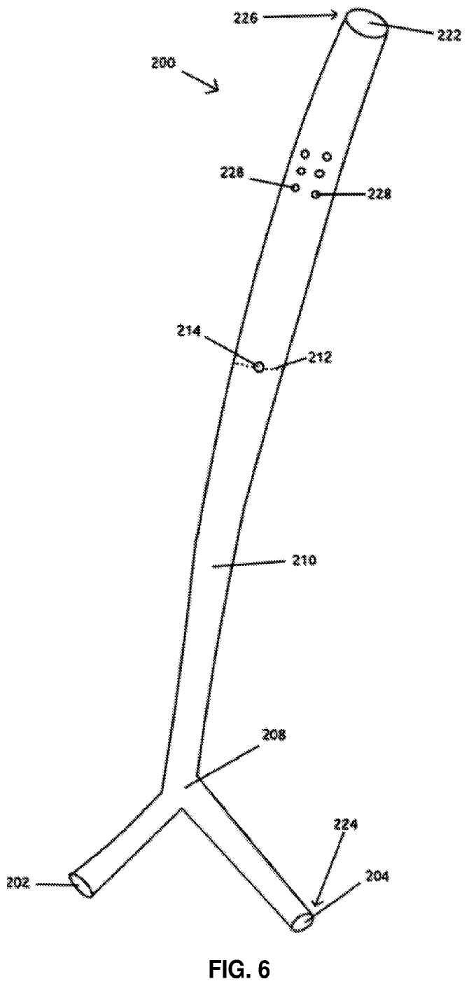

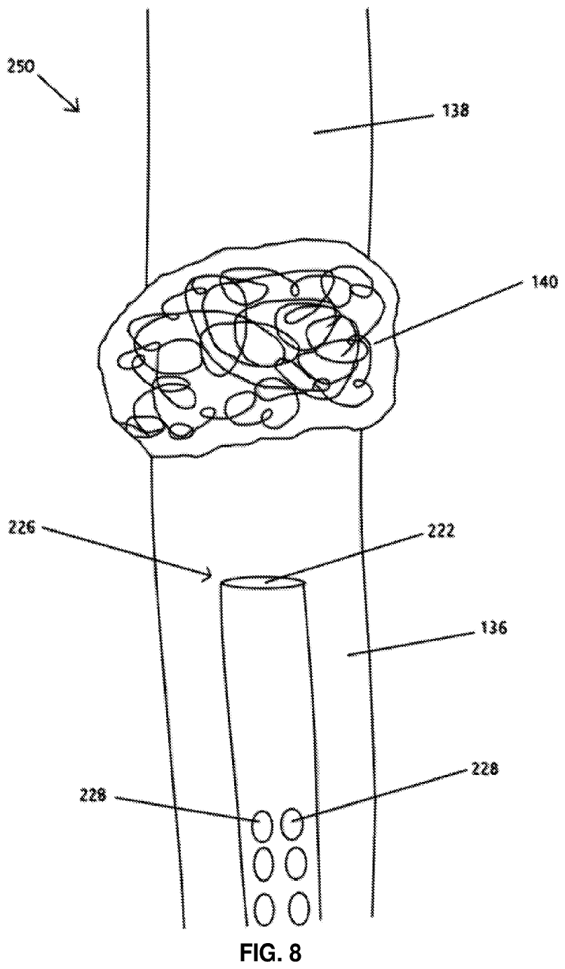

Referring now to FIGS. 6-10, a second embodiment of a bore embolization catheter 200 is depicted. The bore embolization catheter 200 depicted in FIGS. 6-10 is similar to the bore embolization catheter 100 depicted in FIGS. 1-5, except that the bore embolization catheter 200 does not include an inflatable balloon, and therefore has two ports 202, 204 extending proximally from the catheter junction 208 instead of three ports extending proximally from the catheter junction 208.

Referring to FIG. 6, the bore embolization catheter 200 may be a microcatheter, having a catheter main body 210 with a diameter ranging from 0.70 mm to 1.30 mm. However, other sizes are possible and encompassed within the scope of the present disclosure. The bore embolization catheter 200 includes a catheter main body 210 extending from a catheter junction 208 to a distal end 226 of the bore embolization catheter 200. Extending from the catheter junction 208, the bore embolization catheter 200 includes a first port 202 and a second port 204 which extend further proximally. The ports 202, 204 define openings which provide access to internal lumens 272, 274, as discussed in more detail below. The first port 202 and the second port 204 extend proximally from the catheter junction 208, in which the first port 202 and the second port 204 are joined together to form the catheter main body 210. The bore embolization catheter 200 has a proximal end 224 defined by the ends of the first port 202 and the second port 204.

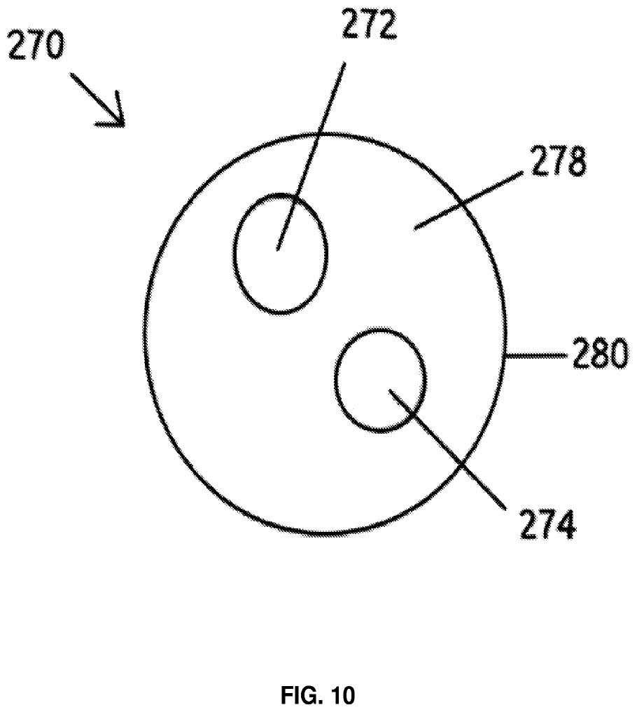

Referring now to FIG. 10, depicted is a sectional view of the catheter main body 210, a perimeter of which is defined by a catheter wall 280. The catheter wall 280 bounds a catheter interior 278, in which a first lumen 272 and a second lumen 274 are disposed. The first port 202 feeds into, and is in communication with, the first lumen 272 within the catheter main body 210. The second port 204 feeds into, and is in communication with, the second lumen 274 within the catheter main body 210. Thus, when a substance is introduced into the first port 202, the substance may flow into the first lumen 272, and when a substance is introduced into the second port 204, the substance may flow into the second lumen 274.

Referring to FIGS. 6-10, the first lumen 272 extends through the catheter main body 210 and feeds into, and is in communication with, one or more agent delivery holes 228. The agent delivery holes 228 are holes in the catheter main body 210 configured to allow passage of a substance therethrough, i.e., from the first lumen 272 out of the catheter main body 210 and into an area adjacent to the bore embolization catheter 210. The agent delivery holes 228 may number from four to eight in some embodiments. In some embodiments, the bore embolization catheter 200 includes six agent delivery holes 228, as this number is useful for deploying an adequate amount of embolic agent 262 in a desired location without the embolic agent 262 spreading too far from the desired location. However, the number of agent delivery holes 228 is not particularly limited, and other numbers of agent delivery holes 228 are possible and encompassed within the scope of the present disclosure. Non-limiting examples of the embolic agent 262 that can be deployed include, but are not limited to, polyvinyl alcohol (PVA), tris-acryl gelatin microspheres, sclerosant, ethanol, polymers, N-butyl cyanoacrylate (NBCA) glue, or ethylene vinyl alcohol. However, other embolic agents, such as a glue substance, a powder substance, or any accepted materials that are normally put into a vessel, may be utilized and delivered through the agent delivery holes 228. The embolic agent 262 can form a proximal plug within the vessel 136.

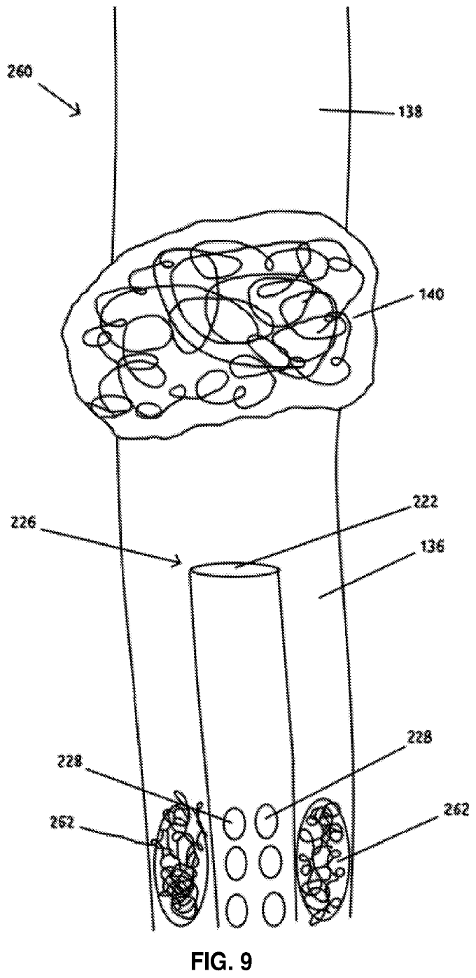

Referring still to FIGS. 6-10, the agent delivery holes 228 are disposed between the catheter junction 208 and the distal end 226. As seen in FIG. 9, the agent delivery holes 228 can be used to deliver an embolic agent 262 to a location within a vessel 136 a distance away from an AVM nidus 140, and in a location such that a treatment can be delivered to the AVM nidus 140 through the third port 122 in a space between the embolic agent 262 and the AVM nidus 140. To deliver an embolic agent 262 through the agent delivery holes 228, the embolic agent 262 can be introduced into the first port 202, and flow through the first lumen 274 and out the agent delivery holes 228 into the vessel 136. Advantageously, this allows for an area within the vessel 136 to be effectively sealed off to prevent backflow of a treatment substance into normal vessels 134.

Referring again to FIGS. 6-10, the second lumen 274 extends through the catheter main body 210 and feeds into, and is in communication with, the end port 222 at the distal end 226 of the bore embolization catheter 200. A medication or other substance useful for treating an AVM nidus 140 or other malformation may be introduced into the second port 204 and flow through the second lumen 274 and out of the end port 222 at the distal end 226 of the bore embolization catheter 200. Thus, the second port 204, second lumen 274, and end port 222 may be used to administer a substance such as a sclerosant solution to an area within a vessel 136 needing treatment, such as an area adjacent to an AVM nidus 140. Non-limiting examples of sclerosant solutions include hypertonic saline (HS), hypertonic dextrose, sodium salicylate, and a combination of HS and hypertonic dextrose. The end port 222 can also be used to deliver any other type of substance that can shrink the vessel 136 and/or cause scars to form. As depicted in FIG. 9, the distal end 226 may be disposed between the AVM nidus 140 and the embolic agent 162 delivered through the agent delivery holes 228, effectively creating a sealed off area to prevent backflow of the treatment substance.

Notwithstanding the above description of the first lumen 272 and the second lumen 274, it is understood that the lumens 272, 274 may be switched or reversed in any configuration. In other words, it is possible, for example, for the first lumen 272 to connect the first port 202 to the end port 222, or for the second lumen 274 to connect the first port 202 to the agent delivery holes 228, and so on. The lumens 272, 274 can also be positioned in any desired arrangement in the catheter interior 278 within the catheter main body 210. Furthermore, though the administration of an embolic agent 262 through the agent delivery holes 228 and the administration of a sclerosant solution through the end port 222 are described for exemplary purposes, the composition of any substance or drug delivered through the bore embolization catheter 200 is not particularly limited.

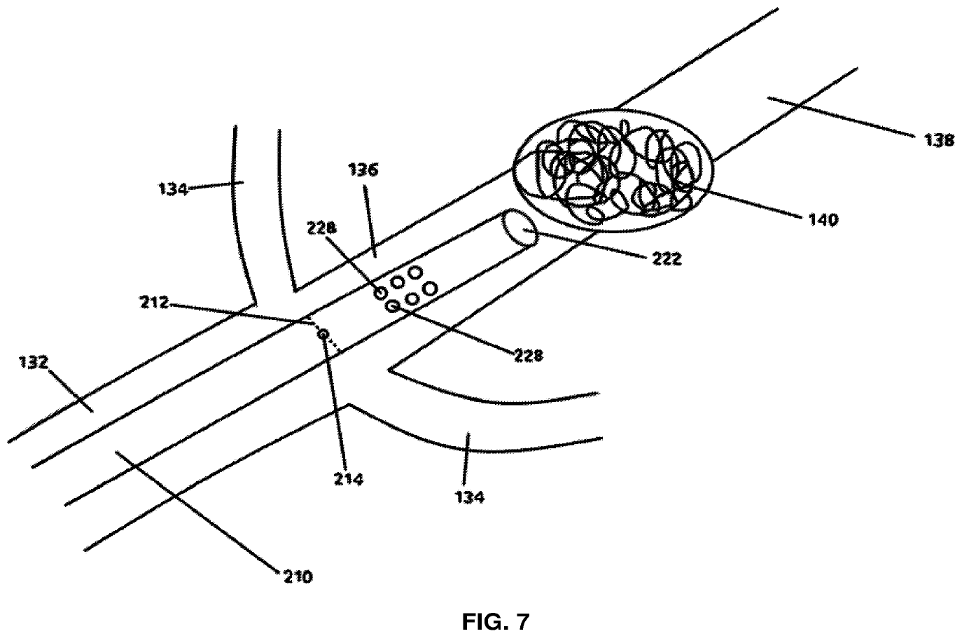

Referring now to FIGS. 6-7, the catheter main body 210 may include a detachment zone 212, which can be in the form of perforations, indentations, or other weak points in the material making up the catheter main body 210, and/or a detachment zone hole 214. The detachment zone 212 and/or the detachment zone hole 214 can be used for the purpose of breaking the catheter main body 210 at a desired location after a procedure has been completed or even during a procedure, as desired. The agent delivery holes 228 and end port 122 are each distal to the detachment zone 212 and detachment hole 214. The detachment zone 212 and/or the detachment zone hole 214 allow for a section of the bore embolization catheter 200 (i.e., the section distal to the detachment zone 212 and/or detachment zone hole 214) to remain in place within a vessel 136 instead of having to remove it. Advantageously, this can prevent inadvertently causing damage to the vessel 136 and to surrounding, normal vessels 134 from the process of the retrieving the bore embolization catheter 200 once the embolic agent 262 has been delivered. In some embodiment, the detachment zone 212 and/or detachment hole 214 may cause breakage of the catheter main body 210 upon applying a twisting and/or pulling motion to the proximal end 224 of the bore embolization catheter 200 once the delivered embolic agent 262 is situated around the agent delivery holes 228. The embolic agent 262 may adhere to the catheter main body 210 sufficiently enough to cause resistance to the movement of the catheter main body 210 in an amount sufficient to cause breakage of the catheter main body 210 at the detachment zone 212 and/or detachment hole 214.

Referring still to FIGS. 6-10, the end port 222 may also be utilized to drain fluids from within a vessel 136, such as from an around an AVM nidus 140 or between an AVM nidus 140 and the delivered embolic agent 262. Fluids can be allowed to enter the end port 222 and travel through the second lumen 274 to the second port 206 (optionally, with the aid of suction applied to the third port 206) in order to drain. Alternatively, if the catheter main body 210 has been broken at the detachment zone 212 and/or detachment hole 214, as described in more detail above, then such fluids may drain out of the broken catheter main body 210 at the break (i.e., at the detachment zone 212 and/or detachment hole 214), which may be on the opposing side of an embolic agent 262 compared to an AVM nidus 140 or other malformation.

As discussed above, each of the first port 202 and the second port 204 is configured to perform separate functions. However, it does not matter which port 202, 204 is used for which function, and it does not matter which lumen 272, 274 connects to a given port 202, 204. Each of the ports 202, 204 may be used for a different function. For example, the first port 202 can be used to administer embolic agent 262 through the agent delivery holes 228, and the second port 204 can be used to deliver a treatment substance, or any other means of treating an AVM nidus 140, through the end port 226. However, this is also not necessary. Rather, in some embodiments, for example, both of the ports 202, 204 may be utilized for delivering the same substance.

Referring now to FIG. 7, the bore embolization catheter 200 is depicted situated within a feeding artery 132 and within the inflow vessel 136. The feeding artery 132 is the vessel through which the bore embolization catheter 200 is fed to reach the location in need of treatment. The bore embolization catheter 200 is depicted situated within the feeding artery 132 and extending past normal vessels 134 into the inflow vessel 136. The inflow vessel 136 is the vessel in which embolic agent 262 is to be delivered. Behind the AVM nidus 140, relative to the bore embolization catheter 200, is the outflow portion 138 of the vessel 136. A portion of the bore embolization catheter 200 that includes the agent delivery hole 228 can be positioned within the inflow vessel 136, such that an embolic agent 262 can be delivered through the agent delivery holes 228, as depicted in FIG. 9, and the embolic agent 262 is present within the inflow vessel 136 and not within any other vessels.

Referring now to FIGS. 7-9, depicted is a method for applying a treatment to an AVM nidus 140 with the bore embolization catheter 200. The bore embolization catheter 200 can be used for treating irregularities or malformations in blood vessels including, but limited to, an AVM nidus 140. The method for treating an AVM nidus 140 can begin by inserting the bore embolization catheter 200 into a feeding artery 132. After inserting the bore embolization catheter 200 into the feeding artery 132, the bore embolization catheter 200 can be guided through the feeding artery 132 into an inflow vessel 136 in which the AVM nidus 140 is located. The bore embolization catheter 200 can then be positioned between the AVM nidus 140 and a normal vessel 134. The embolic agent 262 can then be deployed by administering the embolic agent 262 into the first port 202, through the first lumen 272, and out the agent delivery holes 228. Finally, the treatment substance is deployed to treat the AVM nidus 140 by administering the treatment substance into the second port 204, through the second lumen 274, and out the end port 222. Also, the end port 222 can be used to drain fluids from the distal end 226 of the bore embolization catheter 200. Once the treatment substance has been delivered, the bore embolization catheter 200 may be broken at the detachment zone 212 or detachment hole 214 to leave in place some of the bore embolization catheter 200, with embolic agent 262 around it, and the remaining portion of the bore embolization catheter 200 can be retrieved.

Any of the embodiments of the bore embolization catheter 100, 200 may be modified to include an additional balloon at any location on the main body 110, 210 of the bore embolization catheter 100, 200. The additional balloon may be, for instance, at the most distal end 126, 226 of the bore embolization catheter 100, 200, may be proximal to the agent delivery holes 128, 228 but distal to the detachment zone 112, 212, or may be proximal to the detachment zone 112, 212. In other embodiments, two or more additional balloons are included. It is understood that an additional balloon can be placed at any location on the main body 110, 210 depending on its intended use. The placement of the additional balloon at different points along the main body 110, 210 of the bore embolization catheter 100, 200 can provide various advantages such as, but not limited to, precise substance delivery without unwanted backflow within a vessel or flow to a region of a vessel where the substance is not desired to be delivered. Certain embodiments of the devices and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the devices and methods described herein to various usages and conditions. Various changes may be made, and equivalents may be substituted for elements thereof without departing from the essential scope of the disclosure. In addition, many modifications may be made to adapt a particular situation or material to the teachings of the disclosure without departing from the essential scope thereof.

1. A bore embolization catheter comprising:
a catheter main body extending from a catheter junction to a distal end and housing a first lumen, a second lumen, and a third lumen;
a detachment zone on the catheter main body configured to allow the catheter main body to be broken;
an agent delivery hole on the catheter main body between the detachment zone and the distal end;
an end port at the distal end;
an inflatable balloon on the catheter main body between the agent delivery hole and the distal end; and
a first port, a second port, and a third port extending from the catheter junction and defining a proximal end, wherein the first port is in communication with the inflatable balloon through the first lumen, the second port is in communication with the agent delivery hole through the second lumen, and the third port is in communication with the end port through the third lumen.

2. The bore embolization catheter of claim 1, wherein the bore embolization catheter is a microcatheter, the catheter main body having a diameter ranging from 0.70 mm to 1.30 mm.

3. The bore embolization catheter of claim 1, wherein the catheter junction is between the detachment zone and the proximal end.

4. The bore embolization catheter of claim 1, wherein the detachment zone is between the catheter junction and the agent delivery hole.

5. The bore embolization catheter of claim 1, wherein the agent delivery hole is between the inflatable balloon and the detachment zone.

6. The bore embolization catheter of claim 1, wherein the detachment zone includes a detachment hole.

7. (canceled)

8. The bore embolization catheter of claim 1, comprising six a plurality of agent delivery holes all disposed between the inflatable balloon and the detachment zone.

9. A method of using the bore embolization catheter of claim 1, the method comprising:
inserting the bore embolization catheter into a feeding artery;
guiding the bore embolization catheter through the feeding artery and into an inflow vessel having an AVM nidus located therein;
positioning the bore embolization catheter between the AVM nidus and a normal vessel;
inflating the inflatable balloon within the inflow vessel by applying a fluid through the first port to form an inflated balloon;
delivering an embolic agent to occlude the inflow vessel proximally to the inflated balloon by administering the embolic agent into the second port and through the agent delivery hole;
treating the AVM nidus by administering a treatment substance into the third port and through the end port, wherein the treatment substance is prevented from flowing into the inflow vessel by the embolic agent.

10. The method of claim 9, further comprising draining fluids near the distal end through the end port.

11. The method of claim 9, further comprising breaking the bore embolization catheter at the detachment zone.

12. A bore embolization catheter comprising:
a catheter main body extending from a catheter junction to a distal end and housing a first lumen and a second lumen;
a detachment zone on the catheter main body configured to allow the catheter main body to be broken;
an agent delivery hole between the detachment zone and the distal end;
an end port at the distal end; and
a first port and a second port extending from the catheter junction and defining a proximal end, wherein the first port is in communication with the agent delivery hole through the first lumen, and the second port is in communication with the end port through the second lumen.

13. The bore embolization catheter of claim 12, wherein the bore embolization catheter is a microcatheter, the catheter main body having a diameter ranging from 0.70 mm to 1.30 mm.

14. The bore embolization catheter of claim 12, wherein the catheter junction is between the detachment zone and the proximal end.

15. The bore embolization catheter of claim 12, wherein the detachment zone is between the catheter junction and the agent delivery hole.

16. The bore embolization catheter of claim 12, wherein the agent delivery hole is between the detachment zone and the distal end.

17. The bore embolization catheter of claim 12, wherein the detachment zone includes a detachment hole.

18. (canceled)

19. The bore embolization catheter of claim 12, comprising a plurality of agent delivery holes all disposed between the detachment zone and the distal end.

20. A method of using the bore embolization catheter of claim 12, the method comprising:
inserting the bore embolization catheter into a feeding artery;
guiding the bore embolization catheter into an inflow vessel having an AVM nidus therein;
positioning the bore embolization catheter between the AVM nidus and the normal vessel;
delivering an embolic agent into the first port and out of the agent delivery hole into the inflow vessel; and
delivering a treatment substance to treat the AVM nidus by administering the treatment substance into the second port and out of the end port, wherein the treatment substance is prevented from flowing into the feeding artery by the embolic agent.

21. The method of claim 20, further comprising breaking the bore embolization catheter at the detachment zone.

22. The method of claim 20, further comprising draining fluids near the distal end through the end port.
